(19) United States
(12) Patent Application Publication
(10) Pub. No.: US 2005/0203606 A1
(43) Pub. Date: Sep. 15, 2005

(54) Stent system for preventing restenosis

(76) Inventor: Daniel Henry VanCamp, Covington, WA (US)

Correspondence Address: JONES DAY, 222 EAST 41ST ST, NEW YORK, NY, 10017, US

(21) Appl. No.: 10/797,737

(22) Filed: Mar. 9, 2004

(51) Int. Cl. A61F 2/06

(52) U.S. Cl. 623/1.15

(57) ABSTRACT

A system for treating a body lumen is disclosed. The system comprises an outer stent and an inner stent disposed within the lumen of the outer stent. At least one end of the inner stent extends outside of the lumen of the outer stent, so that the end of the inner stent contacts and conforms to the body lumen wall that is adjacent the end of the outer stent. A coating can be disposed on a surface, preferably the outer surface, of the inner stent. The coating contains a therapeutic substance that may be released into the body lumen wall to help in preventing restenosis. Also disclosed is a stent having a balloon-expandable portion connected to a self-expanding portion. Methods for deploying the system and the stent are also disclosed.

### FIELD OF THE INVENTION

This invention relates generally to stents or systems for treating a body lumen comprising stents. More specifically, this invention is directed to a system to prevent restenosis resulting from damage caused by the deformation of a body lumen wall by a stent, and methods of deploying the same.

### BACKGROUND OF THE INVENTION

The use of stents in the treatment of blood body lumens to aid in the prevention of restenosis (the re-narrowing or closing of a body lumen caused by the overproduction of cells, similar to formation of scar tissue) is well known. Stents are typically delivered in a contracted state to the treatment area within a lumen, where they are then expanded. Balloon-expandable stents expand from a contracted state by deforming in response to a force exerted upon the stent body by a balloon that is inflated within the stent's lumen. Once expanded within a body lumen, the stent body is strong enough to resist any contracting force exerted by the body lumen wall so that the stent maintains its expanded diameter. In contrast, self-expanding stents have resilient bodies that exert a radial expansion force when the stent is compressed. A self-expanding stent that is deployed within a body lumen will expand until the body lumen wall exerts a compressive force against the stent that is equal to the radial expansion force.

The use of balloon-expandable and self-expanding stents, however, may have the disadvantage of causing additional trauma to a body lumen upon deployment of the stent. Typically, as shown in FIG. 1, a stent 100 is expanded within a body lumen 500 so that the diameter of the stent 100 is greater than that of the body lumen 500. As a result, the edges of the ends of stent 100 may be pressed into the wall 510 of body lumen 500, stressing the wall 510 to the point of creating additional trauma. i.e. cutting or tearing of the body lumen wall 510. This trauma may ultimately lead to restenosis in the areas of the body lumen adjacent the ends of the stent.

Recently, various types of drug-coated stents have been used for the localized delivery of drugs to the wall of a body lumen to further prevent restenosis. Although known drug-coated stents may be effective in delivering a therapeutic drug or agent to tissue that is in direct contact with the coating on the outer surface of the stent, this coating may not be effective in delivering therapeutic substances to the areas adjacent the end of the stent that are not in direct contact with the coating. This is especially true of the area of the body lumen that is upstream of the stent.

Therefore, there is a need for a medical device that can deliver a therapeutic substance to the areas of a body lumen wall adjacent to the ends of a stent that is deployed within the body lumen, without causing additional trauma to the body lumen wall. There is also a need for a method of deploying such a device.

### SUMMARY OF THE INVENTION

The present invention addresses the disadvantages discussed above by providing a system that is capable of delivering a therapeutic agent to the areas of a body lumen wall that may have been damaged by the deployment of a first balloon-expandable stent. This is accomplished by deploying a second self-expanding stent within the lumen of the first balloon-expandable stent. Preferably, the second self-expanding stent has a surface, such as an outer surface, and a coating disposed on at least part of the surface. This coating is placed into contact the areas of the body lumen wall adjacent to the edges or ends of the first balloon-expandable stent. The coating contains a therapeutic substance that is capable of being released into the body lumen wall. The second self-expanding stent conforms to the contours of the first stent and the body lumen wall without exerting a force that is sufficient to cause further deformation to the body lumen wall.

In a preferred embodiment, an implantable system for treating a body lumen having a lumen wall comprises (a) an outer balloon-expandable stent comprising a first end, a second end, a surface, and a lumen; and (b) at least one inner self-expanding stent comprising a first end, a second end, and a surface, wherein the inner stent is capable of being deployed so that at least a portion of the inner stent is disposed within the lumen of the outer stent, and the first end of the inner stent is disposed outside the lumen of the outer stent. The second end of the inner stent may be disposed outside the lumen of the outer stent. The outer stent may be capable of exerting a radial force against the body lumen wall that is greater than the radial force that the inner stent is capable of exerting against the body lumen wall. The inner stent may further comprise a coating comprising a biologically active material disposed on at least a part of the surface of the inner stent. The coating may be disposed proximate the first end of the inner stent, or it may be disposed proximate the first end of the inner stent and proximate the second end of the inner stent. The surface of the inner stent may be an outer surface. The coating may further comprise a polymeric material. The biologically active material may comprise pacliltaxel and the coating may further comprise a polymeric material. The outer stent may further comprise a coating comprising a biologically active material disposed on at least a part of the surface of the outer stent. The coating may further comprise a polymeric material. The biologically active material may comprise paclitaxel.

In another preferred embodiment, an implantable system for treating a body lumen having a lumen wall comprises (a) an outer balloon-expandable stent comprising a first end, a second end, a surface, and a lumen; and (b) an inner self-expanding stent comprising a first end, a second end, and a surface, wherein the inner stent is capable of being deployed so that at least a portion of the inner stent is disposed within the lumen of the outer stent, and the first and second ends of the inner stent are disposed outside of the lumen of the outer stent, the inner stent comprises a first coating comprising a first biologically active material disposed on a first part of the surface of the inner stent that is proximate the first end of the inner stent and on a second part of the surface of the inner stent that is proximate the second end of the inner stent, and the outer stent comprises a second coating comprising a second biologically active material disposed on at least a part of the surface of the outer stent.

In another preferred embodiment, an implantable system for treating a body lumen having a lumen wall comprises (a) an outer balloon-expandable stent comprising a first end, a second end, a surface, and a lumen; and (b) a first self-expanding inner stent comprising a first end, a second end, and a surface, wherein the first inner stent is capable of being deployed so that the first end of the first inner stent is disposed outside of the lumen of the outer stent and the second end of the first inner stent is disposed within the lumen of the outer stent. The system may further comprise a second inner self-expanding stent comprising a first end, a second end, and a surface, wherein the second inner stent is capable of being deployed so that the first end of the second inner stent is disposed outside of the lumen of the outer stent and the second end of the second inner stent is disposed within the lumen of the outer stent. The outer stent may be capable of exerting a radial force against the body lumen wall that is greater than the radial force that the first or second inner stent is capable of exerting against the body lumen wall. The first inner stent may comprise a first coating comprising a first biologically active material disposed on at least a part of the surface of the first inner stent. The coating may be proximate the first end of the first inner stent. The second inner stent may comprise a second coating comprising a second biologically active material disposed on at least a part of the surface of the second inner stent. The second coating may be disposed on a part of the surface of the second inner stent that is proximate the first end of the second inner stent. The system may have at least one of the first coating or second coating further comprising a polymeric material. The system may have at least one of the first biologically active material or the second biologically active material comprises pacliltaxel. The outer stent may comprise a third coating comprising a third biologically active material disposed on at least a part of the surface of the outer stent. The third coating may further comprise a polymeric material, and the third biologically active material may comprise paclitaxel. The first coating may also be disposed on the outer surface of the first inner stent and the second coating may be disposed on the outer surface of the second inner stent.

In another preferred embodiment, an implantable system for treating a body lumen having a lumen wall comprises (a) an outer balloon-expandable stent comprising a first end, a second end, a surface, and a lumen; (b) a first inner self-expanding stent comprising a first end, a second end, and a surface; and (c) a second inner self-expanding stent comprising a first end, a second end, and a surface, wherein the first inner stent is capable of being deployed so that the first end of the first inner stent is disposed outside of the lumen of the outer stent and the second end of the first inner stent is disposed within the lumen of the outer stent, the second inner stent is capable of being deployed so that the first end of the second inner stent is disposed outside of the lumen of the outer stent and the second end of the second inner stent is disposed within the lumen of the outer stent, the first inner stent comprises a first coating comprising a first biologically active material disposed on at least a part of the surface of the first inner stent proximate the first end of the first inner stent, the second inner stent comprises a second coating comprising a second biologically active material disposed on at least a part of the surface of the second inner stent proximate the first end of the second inner stent, and the outer stent comprises a third coating comprising a third biologically active material disposed on at least a part of the surface of the outer stent.

In another preferred embodiment, a stent comprises (a) a balloon-expandable portion having a first end and a second end; and (b) a first self-expanding portion having a first end and a second end, wherein the first end of the balloon-expandable portion is connected to the first end of the first self-expanding portion. The stent may further comprise a second self-expanding portion having a first end and a second end, wherein the second end of the balloon-expandable portion is connected to the first end of second self-expanding portion. The balloon-expandable portion may be capable of exerting a radial expansion force against the body lumen wall that is greater than the radial expansion force that the self-expanding portion is capable of exerting against the body lumen wall. The first self-expanding portion may comprise a plurality of wires. The first end of the balloon-expandable portion may be connected to the first end of the first self-expanding portion by weaving the plurality of wires with the first end of the balloon-expandable portion. The plurality of wires may comprise a superelastic material. The first self-expanding portion may further comprise a surface and a coating comprising a biologically active material disposed on at least a part of the surface. The coating may be disposed on a part of the surface that is proximate the second end of the first self-expanding portion. The coating may further comprise a polymeric material. The biologically active material may comprise pacliltaxel. The balloon-expandable portion may further comprise a surface and a coating comprising a biologically active material disposed on at least a part of the surface. The coating may further comprise a polymeric material. The biologically active material may comprise paclitaxel.

In another preferred embodiment, a stent comprises (a) a balloon-expandable portion having a first end and a second end; (b) a first self-expanding portion having a first end and a second end, wherein the first end of the balloon-expandable portion is connected to the first end of the first self-expanding portion; and (c) a second self-expanding portion having a first end and a second end, wherein the second end of the balloon-expandable portion is connected to the first end of second self-expanding portion; wherein the first self-expanding portion comprises a surface and a first coating comprising a first biologically active material disposed on at least a part of the surface of the first self-expanding portion, the second self-expanding portion comprises a surface and a second coating comprising a second biologically active material disposed on at least a part of the surface of the second self-expanding portion, and the balloon-expandable portion comprises a surface and a third coating comprising a third biologically active material disposed on at least a part of the surface of the balloon-expandable portion.

### BRIEF DESCRIPTION OF THE DRAWINGS

FIG. 1 is a cross-sectional view of a balloon-expandable stent deployed within a body lumen.

FIG. 2 is a cross-sectional view of a preferred embodiment of a system in accordance with the present invention.

FIG. 3 is a cross-sectional view of another preferred embodiment of a system in accordance with the present invention.

FIG. 4 is a partial cross-sectional view of a step in a preferred method of deploying a system according to the present invention.

FIG. 5 is a partial cross-sectional view of a preferred embodiment of a delivery member for use with a system of the present invention.

FIG. 6 is a partial cross-sectional view of another preferred embodiment of a delivery member for use with a system of the present invention.

FIG. 7 is a perspective view of another system in accordance with the present invention.

FIG. 8 is a partial side view of the system of FIG. 7.

FIG. 9 is a front cross-sectional view of the stent of FIG. 1.

FIG. 10 is a partial side view of the system of FIG. 2.

### DETAILED DESCRIPTION

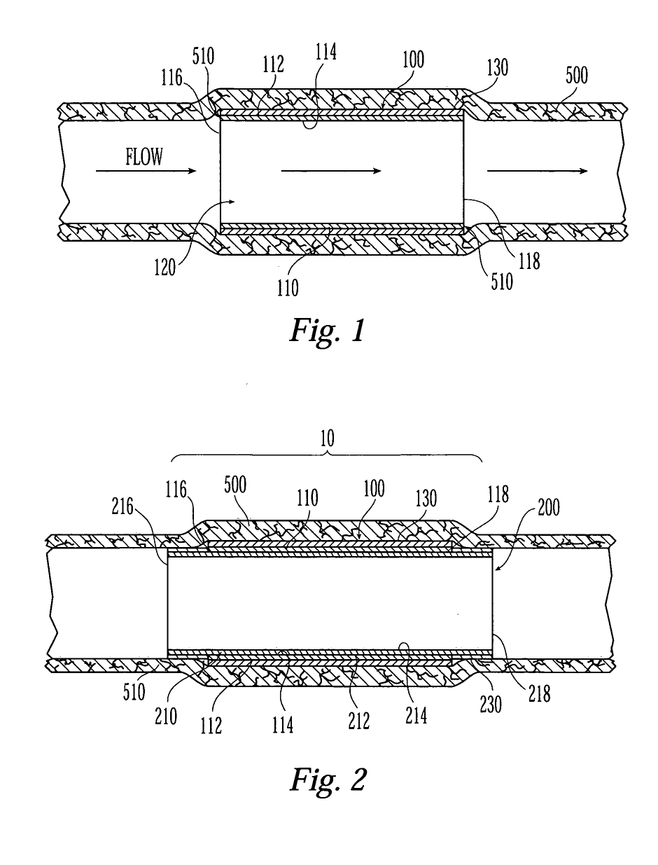

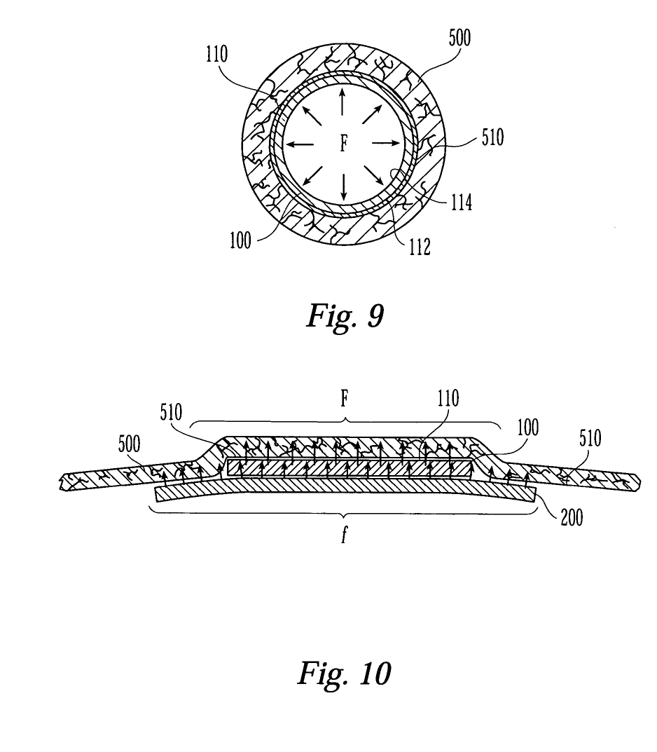

A preferred embodiment of the present invention is illustrated in FIG. 2. System 10 comprises outer balloon-expandable stent 100 and inner self-expanding stent 200. Outer stent 100 may be a stent that is known in the prior art, such as the stent illustrated in FIG. 1. Outer stent 100 comprises body or wall 110 having first end 116, second end 118, outer surface 112, inner surface 114, and lumen 120. Outer stent 100 may further comprise coating 130 disposed on at least a part of a surface of outer stent 100, preferably the outer surface 112. As shown in FIG. 9, body 110 of outer stent 100 exerts a radial force F on the walls of body lumen 500. As used hereinafter, the term radial force will refer to the force that is exerted upon body lumen wall 510 by a stent that has been completely deployed within body lumen 500.

Inner self-expanding stent 200 comprises body or wall 210 having first end 216, second end 218, outer surface 212, and inner surface 214. Inner stent 200 further comprises coating 230 disposed on at least a part of a surface of inner stent 200, preferably the outer surface 212. In a preferred embodiment, coating 230 is disposed on outer surface 212 proximate first end 216 and second end 218. Coating 230 may also be disposed on the entire outer surface of inner stent 200. Hereinafter, the term proximate includes parts or areas at or near the selected location.

As shown in FIG. 2, inner stent 200 is disposed within lumen 120 of outer stent 100 so that first end 216 and second end 218 of inner stent 200 extend from lumen 120 and coating 230 on first end 216 and second end 218 is in contact with the wall of body lumen 500. Inner self-expanding stent 200 exerts a radial force f that allows body 210 to conform to the contours of inner surface 114 of outer stent 100 and body lumen wall 510 without causing further deformation of outer stent 100 or body lumen wall 510. In other words, inner stent 200 is configured such that a minimum of radial force f is applied to body lumen wall 510 by inner stent 200 (see FIG. 10). Only enough radial force f is present so that coating 230 may be put into contact with body lumen wall, without the potential for causing further damage to the body lumen wall along or adjacent to first end 216 or second end 218 of inner stent 200.

The amount of radial force f exerted by inner stent 200 on body lumen wall 510 is dependent upon several factors, including the fully expanded diameter of inner stent 200, the material comprising inner stent 200, and the geometry (for example, the structure and thickness) of stent body 210. Configuring these various properties is well known in the art. For example, inner stent 200 may have stent body 210 with a small thickness, thus reducing the radial force that may be generated by inner stent 200. It is preferable that the radial expansion force exerted by outer stent 100 on body lumen wall 510 is greater than the radial expansion force exerted by inner stent 200.

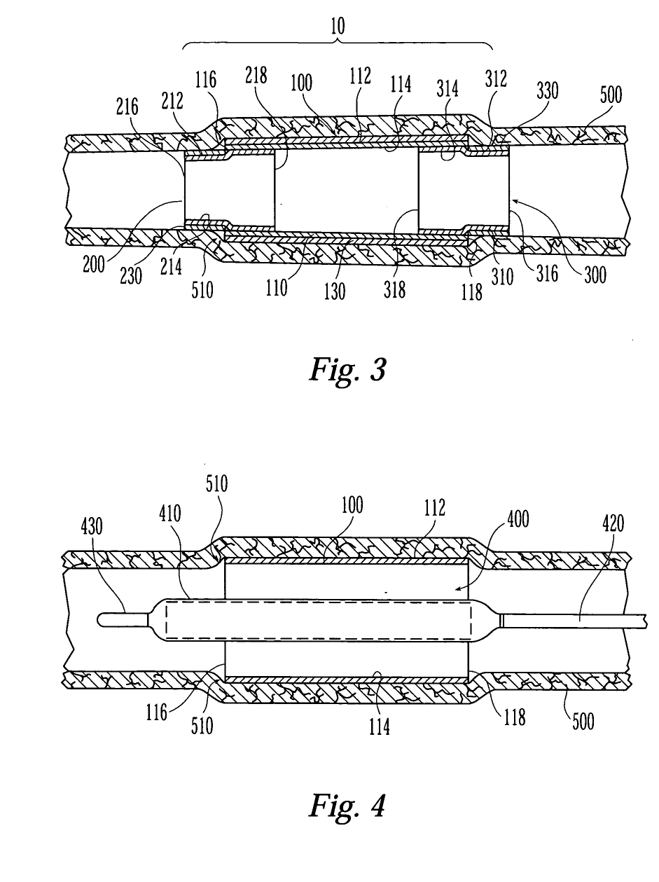

The length and positioning of inner stent 200 in relation to outer stent 100 may be varied according to the needs of the user. As shown in FIG. 2, inner stent 200 may have a length that is greater than the length of outer stent 100, so that first end 216 and second end 218 of inner stent 200 extend out of the lumen 120 of outer stent 100 or beyond first end 116 and second end 118 of outer stent 100, allowing coating 230 (which is proximate ends 216, 218) to contact body lumen wall 510. In a second embodiment, as shown in FIG. 3, only first end 216 of a first inner stent 200 extends from first end 116 of outer stent 100, and second end 218 is disposed within lumen 120. In this embodiment, system 10 may further comprise a second inner stent 300, with first end 316 of second inner stent 300 extending from second end 118 of outer stent 100. This embodiment may be preferable when outer stent 100 is tapered or has a varying diameter, as inner stents 200 and 300 may be configured so that their radial forces may be substantially equal to each other, despite the different diameters of body lumen wall 510 near first end 116 and second end 118 of outer stent 100. FIG. 3 further illustrates how the coating on the inner stents may be disposed in different ways. First inner stent 200 has coating 230 disposed only on a part of its outer surface 212 that is proximate first end 212, while second inner stent 300 has coating 330 disposed along its entire outer surface 312 between its ends 310, 318. Preferably, the coating is disposed on at least a part of the outer surface of the stent that is proximate the end of the stent that extends out of the outer stent lumen. The coatings on the outer and inner stents may comprise the same biologically active material or they may comprise different biologically active materials.

System 10 may be deployed within body lumen 500 by one of several methods. FIG. 4 illustrates a method of deploying inner stent 200 after outer stent 100 is deployed within body lumen 500 by any one of a number of methods well known in the art. Delivery member 400 comprises catheter 420, guide wire 430, and sheath 410. Enclosed within sheath 410 is inner stent 200 in a compressed state. Guidewire 410 is guided through body lumen 500 and lumen 120 of outer stent 100. Catheter 400 is then guided over guidewire 410 so that sheath 410 is disposed within lumen 120. Sheath 410 is then removed, allowing inner stent 200 to expand until outer surface 212 contacts body lumen wall 510 and/or inner surface 114 of outer stent 100. This process may then be repeated if more than one inner stent is being used, such as the system of FIG. 3. As discussed above, outer stent 100 and inner stent 200 may have coatings on their surfaces comprising biologically active materials. The coatings may be disposed on either a portion or on the entire surface of a stent, and the coatings on the outer and inner stents may be the same or different from each other.

In addition to preventing the onset of restenosis, system 10 may be used to treat restenosis that has already been diagnosed in the areas adjacent to the ends of previously deployed stents. It may readily be seen that inner stent 200 may be deployed within a stent that was deployed in a previous, separate procedure. Thus, system 10 may be used in situations where it was not previously contemplated or available to be used.

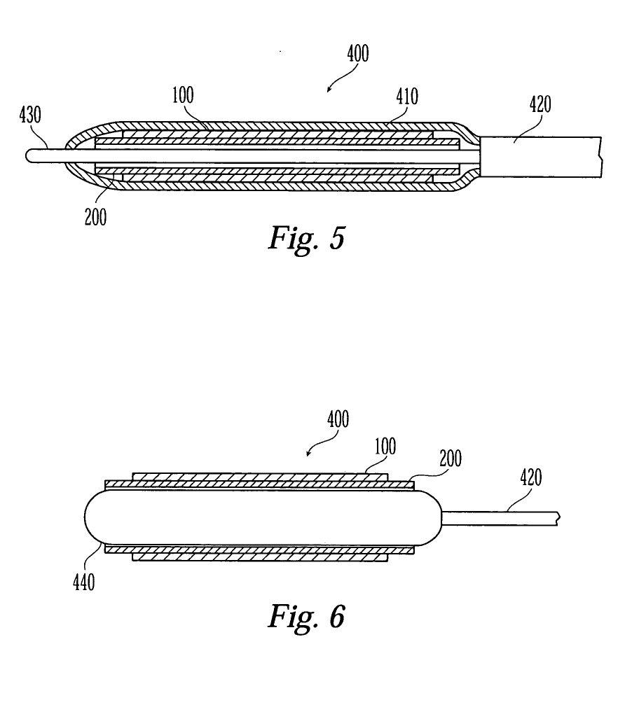

Outer stent 100 and inner stent 200 may also be deployed simultaneously. In a preferred embodiment, both outer stent 100 and inner stent 200 are disposed in a compressed state within sheath 400 of delivery member 410, as shown in FIG. 5. This embodiment may be used when both outer stent 100 and inner stent 200 are self-expanding After release from sheath 100, both outer stent 100 and inner stent 200 expand from their compressed states. Outer stent 100, having a greater radial force, will continue to expand even after contact with body lumen wall 510. Inner stent 200, with a lesser radial force, will expand until it conforms to the contour of inner surface 114 and body lumen wall 510.

In a second preferred embodiment, as illustrated in FIG. 6, outer stent 100 and inner stent 200 may be disposed coaxially with delivery member 400 comprising catheter 420 and balloon 440. In FIG. 6, outer stent 100 and inner stent 200 are in a compressed state, and balloon 440 is in a non-inflated state. This embodiment may be used when outer stent 100 is balloon expandable, and inner stent 200 is self-expanding. In its compressed state, outer stent 100 prevents inner stent 200 from expanding during delivery of the stents into body lumen 500. Balloon catheter is then inflated, expanding body 110 of outer stent 100 through permanent deformation until outer stent 100 reaches the desired diameter. Inner stent 200 will also be expanded by balloon 440 at the same time outer stent 100 is expanded. But because body 210 of inner stent 200 is resilient, the expansion by balloon 400 will not deform it permanently. Thus, when balloon is deflated, body 210 of inner stent 200 will conform itself to the contour of outer stent 200 and body lumen wall 510.

Although radial force f exerted by inner stent body 200 should be kept to a minimum, radial force f should be sufficient to anchor inner stent 200 in place within outer stent lumen 120. This anchoring may be improved by having outer stent inner surface 114 and/or inner stent outer surface 212 further comprise projections or have a surface texture that increase the ability of the two surfaces to interact with each other. Adhesive may also be used to adhesively connect the two stents together.

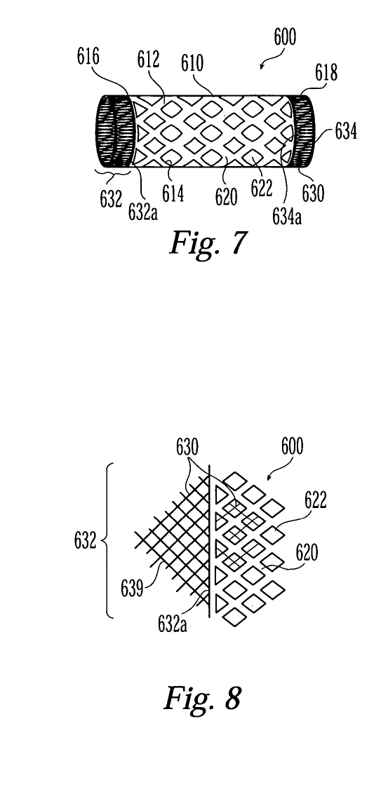

In another embodiment, as illustrated in FIGS. 7 and 8, system 10 may comprise stent 600 having balloon-expandable portion 610 having first and second ends 616, 618. Such portion 110 has an outer surface 612 and inner surface 614. The stent 600 further comprises a plurality of struts 620 and open cells 620 disposed between struts 620.

In this embodiment, a plurality of threads 630 extend from first end 616 and second end 618 to form a first self-expanding portion 632 and a second self-expanding portion 634. Threads 630 are formed of a super elastic material that allow threads 630 to be connected or attached to ends 616 and 618 by weaving threads 630 through struts 620 and cells 622. For example alloys such as Fe/Pt and Fe/Pd alloys exhibit superelastic qualities and may be used to form threads 630. Threads 630 may also be connected or attached to ends 616 and 618 by other methods, such as welding or the use of adhesive. Threads 630 are configured to form a mesh which makes up the self-expanding portions of the stent 632, 634 that is adjacent to first end 616 and second end 618. Mesh or self-expanding portions 632, 634 may exhibit the same self-expanding properties as inner stent 200. Also, the mesh self-expanding portions 632, 634 may first be formed and then the ends of the self-expanding portions 632, 634 are connected to the balloon-expandable portion 610. More specifically, with reference to FIGS. 7 and 8, self-expanding portions 632, 634 may first be formed. The ends 632a, 634a of these self-expanding portions 632, 634 are then connected to the ends 616, 618 of the balloon-expandable portion 610. Alternatively, the thread that makes up the self-expanding portions 632, 634 can be connected to the balloon expandable portion 610 before or while the self-expanding portions 632, 634 are being formed. In this case, the ends 632a, 634a of the self-expanding portions 632, 634 are made up of the parts of the threads or wires that are connected to the balloon-expandable portion 610. Although the self-expanding portions 632, 634 can be a mesh of threads or wires, such self-expanding portions 632, 634 can have other configurations as well. For example, self-expanding portions 632, 634 may be a pattern of struts that is formed by laser-cutting or other methods. Threads 630 of the self-expanding portions may be coated with a therapeutic coating 634. When stent 600 is deployed within a body lumen, self-expanding portions 632, 634 conform to the body lumen wall in a manner similar to that of inner stent 200 described above. Thus, coating 634 contacts the areas of the body lumen wall that are adjacent to first end 616 and second end 618, allowing coating to release therapeutic substances into the body lumen wall.

Outer stent 100 and inner stent 200 may be fabricated from metallic, ceramic, or polymeric materials, or combinations thereof. The material may be porous or nonporous. Porous structural elements can be microporous, nanoporous or mesoporous. Preferred materials are metallic. Suitable metallic materials include metals and alloys based on titanium (such as nitinol, nickel titanium alloys, thermo-memory alloy materials), stainless steel, tantalum, nickel-chrome, or certain cobalt alloys including cobalt-chromium-nickel alloys such as Elgiloy® and Phynox®. The components may also include parts made from other metals such as, for example, gold, platinum, or tungsten. Metallic materials also include clad composite filaments, such as those disclosed in WO 94/16646.

Suitable ceramic materials include, but are not limited to, oxides of the transition elements such as titanium oxides, hafnium oxides, iridium oxides, chromium oxides, and aluminum oxides. Silicon based materials may also be used.

The polymer(s) useful for forming the components of the medical devices should be ones that are biocompatible and avoid irritation to body tissue. The polymers can be either biostable or bioabsorbable. Suitable polymeric materials include without limitation polyurethane and its copolymers, silicone and its copolymers, ethylene vinyl-acetate, polyethylene terephtalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, and chitins.

Other polymers that are useful include, without limitation, dacron polyester, poly(ethylene terephthalate), polycarbonate, polymethylmethacrylate, polypropylene, polyalkylene oxalates, polyvinylchloride, polyurethanes, polysiloxanes, nylons, poly(dimethyl siloxane), polycyanoacrylates, polyphosphazenes, poly(amino acids), ethylene glycol I dimethacrylate, poly(methyl methacrylate), poly(2-hydroxyethyl methacrylate), polytetrafluoroethylene poly(HEMA), polyhydroxyalkanoates, polytetrafluorethylene, polycarbonate, poly(glycolide-lactide) co-polymer, polylactic acid, poly(γ-caprolactone), poly(γ-hydroxybutyrate), polydioxanone, poly(γ-ethyl glutamate), polyiminocarbonates, poly(ortho ester), polyanhydrides, alginate, dextran, chitin, cotton, polyglycolic acid, polyurethane, or derivatized versions thereof, i.e., polymers which have been modified to include, for example, attachment sites or cross-linking groups, e.g., RGD, in which the polymers retain their structural integrity while allowing for attachment of cells and molecules, such as proteins, nucleic acids, and the like.

Outer stent 100 may be fabricated of the same or different material than that of inner stent 200.

As described above, coating 130, 230 may be disposed on a surface, such as the outer surfaces 112, 212 of outer stent 100 and/or inner stent 200. In one method of forming the aforementioned coating layer, a coating material composition is applied to the surface. Coating compositions may be applied by any method to a surface of a stent or medical device to form a coating layer. Examples of suitable methods include, but are not limited to, spraying such as by conventional nozzle or ultrasonic nozzle, dipping, rolling, electrostatic deposition, and a batch process such as air suspension, pan coating or ultrasonic mist spraying. Also, more than one coating method may be used. Coating compositions suitable for applying a coating to the stents of the present invention may include a polymeric material dispersed or dissolved in a solvent suitable for the stent, wherein upon applying the coating composition to the stent, the solvent is removed. Such methods are commonly known to the skilled artisan.

The polymeric material should be a material that is biocompatible and avoids irritation to body tissue. Preferably the polymeric materials used in the coating composition of the present invention are selected from the following: polyurethanes, silicones (e.g., polysiloxanes and substituted polysiloxanes), and polyesters. Also preferable as a polymeric material are styrene-isobutylene-styrene copolymers. Other polymers that may be used include ones that may be dissolved and cured or polymerized on the stent or polymers having relatively low melting points that can be blended with biologically active materials. Additional suitable polymers include thermoplastic elastomers in general, polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, polyethers, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, EPDM (ethylene-propylene-diene) rubbers, fluorosilicones, polyethylene glycol, polysaccharides, phospholipids, and combinations of the foregoing.

Preferably, polymeric materials should be selected from elastomeric polymers such as silicones (e.g., polysiloxanes and substituted polysiloxanes), polyurethanes, thermoplastic elastomers, ethylene vinyl acetate copolymers, polyolefin elastomers, and EPDM rubbers. Because of the elastic nature of these polymers, the coating composition is capable of undergoing deformation under the yield point when the stent is subjected to forces, stress or mechanical challenge.

Solvents used to prepare coating compositions include ones which can dissolve or suspend the polymeric material in solution. Examples of suitable solvents include, but are not limited to, tetrahydrofuran, methylethylketone, chloroform, toluene, acetone, isooctane, 1,1,1,-trichloroethane, dichloromethane, isopropanol, IPA, and mixtures thereof.

The coating layer on the stent may also contain a biological active material. The term “biologically active material” encompasses therapeutic agents, such as biologically active agents, and also genetic materials and biological materials. The genetic materials mean DNA or RNA, including, without limitation, of DNA/RNA encoding a useful protein stated below, intended to be inserted into a human body including viral vectors and non-viral vectors. Viral vectors include adenoviruses, gutted adenoviruses, adeno-associated virus, retroviruses, alpha virus (Semliki Forest, Sindbis, etc.), lentiviruses, herpes simplex virus, ex vivo modified cells (e.g., stem cells, fibroblasts, myoblasts, satellite cells, pericytes, cardiomyocytes, skeletal myocytes, macrophage), replication competent viruses (e.g., ONYX-015), and hybrid vectors. Non-viral vectors include artificial chromosomes and mini-chromosomes, plasmid DNA vectors (e.g., pCOR), cationic polymers (e.g., polyethyleneimine, polyethyleneimine (PEI)) graft copolymers (e.g., polyether-PEI and polyethylene oxide-PEI), neutral polymers PVP, SP1017 (SUPRATEK), lipids or lipoplexes, nanoparticles and microparticles with and without targeting sequences such as the protein transduction domain (PTD). The biological materials include cells, yeasts, bacteria, proteins, peptides, cytokines and hormones. Examples for peptides and proteins include growth factors (FGF, FGF-1, FGF-2, VEGF, Endotherial Mitogenic Growth Factors, and epidermal growth factors, transforming growth factor and platelet derived endothelial growth factor, platelet derived growth factor, tumor necrosis factor, hepatocyte growth factor and insulin like growth factor), transcription factors, proteinkinases, CD inhibitors, thymidine kinase, and bone morphogenic proteins (BMP's), such as BMP-2, BMP-3, BMP-4, BMP-5, BMP-6 (Vgr-1), BMP-7 (OP-1), BMP-8. BMP-9, BMP-10, BMP-11, BMP-12, BMP-13, BMP-14, BMP-15, and BMP-16. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells may be of human origin (autologous or allogeneic) or from an animal source (xenogeneic), genetically engineered, if desired, to deliver proteins of interest at the transplant site. The delivery media can be formulated as needed to maintain cell function and viability. Cells include whole bone marrow, bone marrow derived mono-nuclear cells, progenitor cells (e.g., endothelial progentitor cells) stem cells (e.g., mesenchymal, hematopoietic, neuronal), pluripotent stem cells, fibroblasts, macrophage, and satellite cells.

Biologically active material also includes non-genetic therapeutic agents, such as:
anti-thrombogenic agents such as heparin, heparin derivatives, urokinase, and PPack (dextrophenylalanine proline arginine chloromethylketone);
anti-proliferative agents such as enoxaprin, angiopeptin, or monoclonal antibodies capable of blocking smooth muscle cell proliferation, hirudin, and acetylsalicylic acid, amlodipine and doxazosin;
anti-inflammatory agents such as glucocorticoids, betamethasone, dexamethasone, prednisolone, corticosterone, budesonide, estrogen, sulfasalazine, and mesalamine;
antineoplastic/antiproliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives;
anesthetic agents such as lidocaine, bupivacaine, and ropivacaine;
anti-coagulants such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory biologically active agent), dipyridamole, protamine, hirudin, prostaglandin inhibitors, platelet inhibitors and tick antiplatelet peptides;
vascular cell growth promotors such as growth factors, Vascular Endothelial Growth Factors (FEGF, all types including VEGF-2), growth factor receptors, transcriptional activators, and translational promotors;
vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
cholesterol-lowering agents; vasodilating agents; and agents which interfere with endogenous vasoactive mechanisms;
anti-oxidants, such as probucol;
antibiotic agents, such as penicillin, cefoxitin, oxacillin, tobranycin
angiogenic substances, such as acidic and basic fibrobrast growth factors, estrogen including estradiol (E2), estriol (E3) and 17-Beta Estradiol; and
biologically active agents for heart failure, such as digoxin, beta-blockers, angiotensin-converting enzyme (ACE) inhibitors including captopril and enalopril.

The biologically active material may also be applied with a coating composition. Coating compositions suitable for applying biologically active materials to the devices of the present invention preferably include a polymeric material and a biologically active material dispersed or dissolved in a solvent which does not alter or adversely impact the therapeutic properties of the biologically active material employed. Suitable polymers and solvents include, but are not limited to, those listed above.

Coating compositions may be used to apply one type of biologically active material or a combination of biologically active materials. In general, the coating layer may be applied as one homogeneous layer, however, the coating layer may be composed of a plurality of layers comprised of different materials. If the coating layer is composed of a plurality of layers, each layer may contain a single biologically active material or a combination of biologically active materials.

It is to be appreciated that the present invention may also comprise a coating having other materials that have a therapeutic effect, such as iridium oxide.

It should be appreciated that the features and components described herein may be used singly or in any combination thereof. Moreover, the present invention is not limited to only the embodiments specifically described herein, and may be used with medical devices other than stents. The disclosed system may be used to deliver a therapeutic agent to various types of body lumina, including but not limited to the esophagus, urinary tract, and intestines. The description contained herein is for purposes of illustration and not for purposes of limitation. Changes and modifications may be made to the embodiments of the description and still be within the scope of the invention. Furthermore, obvious changes, modifications or variations will occur to those skilled in the art. Also, all references cited above are incorporated herein, in their entirety, for all purposes related to this disclosure.

While the foregoing description and drawings may represent preferred embodiments of the present invention, it should be understood that various additions, modifications, and substitutions may be made therein without departing from the spirit and scope of the present invention as defined in the accompanying claims. In particular, it will be clear to those skilled in the art that the present invention may be embodied in other specific forms, structures, arrangements, and proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being indicated by the appended claims and not limited to the foregoing description.

1. An implantable system for treating a body lumen having a lumen wall comprising:
(a) an outer balloon-expandable stent comprising a first end, a second end, a surface, and a lumen; and
(b) at least one inner self-expanding stent comprising a first end, a second end, and a surface;
wherein the inner stent is capable of being deployed so that:
at least a portion of the inner stent is disposed within the lumen of the outer stent; and
the first end of the inner stent is disposed outside the lumen of the outer stent.

2. The system of claim 1 wherein the second end of the inner stent is disposed outside the lumen of the outer stent.

3. The system of claim 1 wherein the outer stent is capable of exerting a radial force against the body lumen wall that is greater than the radial force that the inner stent is capable of exerting against the body lumen wall.

4. The system of claim 1 wherein the inner stent further comprises a coating comprising a biologically active material disposed on at least a part of the surface of the inner stent.

5. The system of claim 4 wherein the coating is disposed proximate the first end of the inner stent.

6. The system of claim 4 wherein the coating is disposed proximate the first end of the inner stent and proximate the second end of the inner stent.

7. The system of claim 4 wherein the surface of the inner stent is an outer surface.

8. The system of claim 4 wherein the coating further comprises a polymeric material.

9. The system of claim 4 wherein the biologically active material comprises pacliltaxel and the coating further comprises a polymeric material.

10. The system of claim 1 wherein the outer stent further comprises a coating comprising a biologically active material disposed on at least a part of the surface of the outer stent.

11. The system of claim 10 wherein the coating further comprises a polymeric material.

12. The system of claim 11 wherein the biologically active material comprises paclitaxel.

13. An implantable system for treating a body lumen having a lumen wall comprising:
(a) an outer balloon-expandable stent comprising a first end, a second end, a surface, and a lumen; and
(b) an inner self-expanding stent comprising a first end, a second end, and a surface;
wherein:
the inner stent is capable of being deployed so that at least a portion of the inner stent is disposed within the lumen of the outer stent; and the first and second ends of the inner stent are disposed outside of the lumen of the outer stent;
the inner stent comprises a first coating comprising a first biologically active material disposed on a first part of the surface of the inner stent that is proximate the first end of the inner stent and on a second part of the surface of the inner stent that is proximate the second end of the inner stent; and
the outer stent comprises a second coating comprising a second biologically active material disposed on at least a part of the surface of the outer stent.

14. An implantable system for treating a body lumen having a lumen wall comprising:
(a) an outer balloon-expandable stent comprising a first end, a second end, a surface, and a lumen; and
(b) a first self-expanding inner stent comprising a first end, a second end, and a surface;
wherein the first inner stent is capable of being deployed so that the first end of the first inner stent is disposed outside of the lumen of the outer stent and the second end of the first inner stent is disposed within the lumen of the outer stent.

15. The system of claim 14 further comprising a second inner self-expanding stent comprising a first end, a second end, and a surface;
wherein the second inner stent is capable of being deployed so that the first end of the second inner stent is disposed outside of the lumen of the outer stent and the second end of the second inner stent is disposed within the lumen of the outer stent.

16. The system of claim 15 wherein the outer stent is capable of exerting a radial force against the body lumen wall that is greater than the radial force that the first or second inner stent is capable of exerting against the body lumen wall.

17. The system of claim 14 wherein the first inner stent comprises a first coating comprising a first biologically active material disposed on at least a part of the surface of the first inner stent.

18. The system of claim 17 wherein the coating is proximate the first end of the first inner stent.

19. The system of claim 17 wherein the second inner stent comprises a second coating comprising a second biologically active material disposed on at least a part of the surface of the second inner stent.

20. The system of claim 19 wherein the second coating is disposed on a part of the surface of the second inner stent that is proximate the first end of the second inner stent.

21. The system of claim 19 wherein at least one of the first coating or second coating further comprises a polymeric material.

22. The system of claim 19 wherein at least one of the first biologically active material or the second biologically active material comprises pacliltaxel.

23. The system of claim 15 wherein the outer stent comprises a third coating comprising a third biologically active material disposed on at least a part of the surface of the outer stent.

24. The system of claim 23 wherein the third coating further comprises a polymeric material.

25. The system of claim 24 wherein the third biologically active material comprises paclitaxel.

26. The system of claim 19, wherein the first coating is disposed on the outer surface of the first inner stent and the second coating is disposed on the outer surface of the second inner stent.

27. An implantable system for treating a body lumen having a lumen wall comprising:
(a) an outer balloon-expandable stent comprising a first end, a second end, a surface, and a lumen;
(b) a first inner self-expanding stent comprising a first end, a second end, and a surface; and
(c) a second inner self-expanding stent comprising a first end, a second end, and a surface;
wherein:
the first inner stent is capable of being deployed so that the first end of the first inner stent is disposed outside of the lumen of the outer stent and the second end of the first inner stent is disposed within the lumen of the outer stent;
the second inner stent is capable of being deployed so that the first end of the second inner stent is disposed outside of the lumen of the outer stent and the second end of the second inner stent is disposed within the lumen of the outer stent;
the first inner stent comprises a first coating comprising a first biologically active material disposed on at least a part of the surface of the first inner stent proximate the first end of the first inner stent;
the second inner stent comprises a second coating comprising a second biologically active material disposed on at least a part of the surface of the second inner stent proximate the first end of the second inner stent; and
the outer stent comprises a third coating comprising a third biologically active material disposed on at least a part of the surface of the outer stent.

28. A stent comprising:
(a) a balloon-expandable portion having a first end and a second end; and
(b) a first self-expanding portion having a first end and a second end, wherein the first end of the balloon-expandable portion is connected to the first end of the first self-expanding portion.

29. The stent of claim 28 further comprising a second self-expanding portion having a first end and a second end, wherein the second end of the balloon-expandable portion is connected to the first end of second self-expanding portion

30. The stent of claim 28 wherein the balloon-expandable portion is capable of exerting a radial expansion force against the body lumen wall that is greater than the radial expansion force that the self-expanding portion is capable of exerting against the body lumen wall.

31. The stent of claim 28 wherein the first self-expanding portion comprises a plurality of wires.

32. The stent of claim 32 wherein the first end of the balloon-expandable portion is connected to the first end of the first self-expanding portion by weaving the plurality of wires with the first end of the balloon-expandable portion.

33. The stent of claim 32 wherein the plurality of wires comprises a superelastic material.

34. The stent of claim 28 wherein the first self-expanding portion further comprises a surface and a coating comprising a biologically active material disposed on at least a part of the surface.

35. The stent of claim 34 wherein the coating is disposed on a part of the surface that is proximate the second end of the first self-expanding portion.

36. The stent of claim 34 wherein the coating further comprises a polymeric material.

37. The stent of claim 34 wherein the biologically active material comprises pacliltaxel.

38. The system of claim 28 wherein the balloon-expandable portion further comprises a surface and a coating comprising a biologically active material disposed on at least a part of the surface.

39. The system of claim 38 wherein the coating further comprises a polymeric material.

40. The system of claim 39 wherein the biologically active material comprises paclitaxel.

41. A stent comprising:
(a) a balloon-expandable portion having a first end and a second end;
(b) a first self-expanding portion having a first end and a second end, wherein the first end of the balloon-expandable portion is connected to the first end of the first self-expanding portion; and
(c) a second self-expanding portion having a first end and a second end, wherein the second end of the balloon-expandable portion is connected to the first end of second self-expanding portion;
wherein:
the first self-expanding portion comprises a surface and a first coating comprising a first biologically active material disposed on at least a part of the surface of the first self-expanding portion;
the second self-expanding portion comprises a surface and a second coating comprising a second biologically active material disposed on at least a part of the surface of the second self-expanding portion; and
the balloon-expandable portion comprises a surface and a third coating comprising a third biologically active material disposed on at least a part of the surface of the balloon-expandable portion.
